SEQUENCE LISTING (10 sequences)

SEQ ID NO: 1 - DNA, 23 bp, synthetic construct
  source 1..23: mol_type = other DNA
agcttcaggatctcatgcccatc

SEQ ID NO: 2 - DNA, 23 bp, synthetic construct
  source 1..23: mol_type = other DNA
ctccgcttctcgtccctcatgga

SEQ ID NO: 3 - DNA, 20 bp, synthetic construct
  source 1..20: mol_type = other DNA
ggggcctcagactgggaatc

SEQ ID NO: 4 - DNA, 23 bp, synthetic construct
  source 1..23: mol_type = other DNA
tcgccttctatcgccttcttgac

SEQ ID NO: 5 - DNA, 20 bp, synthetic construct
  source 1..20: mol_type = other DNA
ggcagaagtggacaccccaa

SEQ ID NO: 6 - DNA, 21 bp, synthetic construct
  source 1..21: mol_type = other DNA
catggagctcaggatgaacgc

SEQ ID NO: 7 - DNA, 20 bp, synthetic construct
  source 1..20: mol_type = other DNA
agcactgaggaaagctggtc

SEQ ID NO: 8 - DNA, 20 bp, synthetic construct
  source 1..20: mol_type = other DNA
cagaccgtgggttcttcaca

SEQ ID NO: 9 - DNA, 20 bp, synthetic construct
  source 1..20: mol_type = other DNA
cgcttccttacctggttgat

SEQ ID NO: 10 - DNA, 18 bp, synthetic construct
  source 1..18: mol_type = other DNA
tccctctccggaatcgaa

(19) United States
(12) Patent Application Publication
(10) Pub. No.: US 2023/0263826 A1
(43) Pub. Date: Aug. 24, 2023

(54) Compositions and Methods for Treatment and Prevention of Type 1 Diabetes

(71) Applicant: The UAB Research Foundation, Birmingham, AL (US)

(72) Inventors: Sasanka Ramanadham, Birmgham, AL (US); Hanil Kumar Challa, Birmingham, AL (US)

(21) Appl. No.: 17/891,020

(22) Filed: Aug. 18, 2022

Related U.S. Application Data
(60) Provisional application No. 63/234,652, filed on Aug. 18, 2021.

(51) Int. Cl. A61K 35/17; A61P 3/08

(52) U.S. Cl. CPC A61K 35/17; A61P 3/08

(57) ABSTRACT

The invention relates to cells for adoptive cell therapy, e.g., adoptive immunotherapy. The cells include macrophages in which the iPLA₂β gene is disrupted. Also provided are methods and uses of the cells, such as in adoptive therapy in the treatment or prevention of type 1 diabetes (T1D). Also provided are methods for engineering, preparing, and producing the cells, and compositions containing the cells. In some aspects, the provided embodiments provide an improved composition and method for the treatment of T1D. Among the cells disclosed herein are those in which certain genes and/or gene products have been disrupted, modified and/or repressed, in particular via disruption that impairs or reduces expression of iPLA₂β.

### CROSS-REFERENCE TO RELATED APPLICATION

This application claims priority to U.S. Application No. 63/234,652, entitled “Combination CRISPR-Immunotherapy to Counter T1D” and filed on Aug. 18, 2021 (abandoned), which is incorporated herein by reference.

### SEQUENCE LISTING

An electronic sequence listing (SL 803258-00189.xml; size 9.69 KB; date of creation May 10, 2023) submitted herewith is incorporated by reference in its entirety.

### BACKGROUND TO THE INVENTION

Type 1 diabetes (T1D) is a consequence of autoimmune destruction of β cells, involving activation of cellular immunity and inflammation initiated by early-stage immune cell infiltration of islets. While the roles of various stressors (i.e., cytokines, ROS, glucose) in this process have been studied extensively, the impact of lipids on β cell health during T1D development has not received significant attention. As such, there exists a significant gap in the understanding of how lipids generated by immune cells and/or β cells contribute to β cell demise.

Phospholipases A₂ (PLA₂s) hydrolyze the sn-2 substituent of glycerophospholipids to release a lysophospholipid and a free fatty acid. When the fatty acid is arachidonic acid, it can be metabolized by cyclooxygenases (COX), lipoxygenases (LOX), and cytochrome P450 (CYP) enzymes to generate oxidized bioactive lipids, or eicosanoids, which manifest a variety of effects. Some of the most potent inflammatory eicosanoids are prostaglandin E₂ (PGE₂), leukotrienes (LTs), HETEs, and dihydroxyeicosatrienoic acids (DHETs), and they contribute to autoimmune diseases.

Among the PLA₂s is a Ca²⁺-independent phospholipase A₂ (iPLA₂β), and its activity promotes deleterious outcomes in experimental and clinical diabetes. Immune cells express iPLA₂β, and inhibition of iPLA₂β reduces generation of ROS, as well as antibody production from B cells and TNF-α from CD4⁺ T cells and macrophages (Mϕ). Inhibition of iPLA₂β has been shown to be effective in countering autoimmunity and inflammation. Islet-resident Mϕ and early islet-infiltrating Mϕ promote infiltration of other immune cells, with M1 proinflammatory Mϕ recognized as causative factors in T1D development, whereas M2 antiinflammatory MΦ are protective against T1D .

The lipidome and the exact role, if any, for iPLA2β in T1D remains unclear, however. As such, a need remains for further investigation. In view of these observations, Applicants used lipidomics to gain insight into the lipidome associated with T1D development in NOD mice (hereafter referred to as NOD) and humans at high risk for developing T1D.

### SUMMARY OF THE INVENTION

The present disclosure relates to an engineered macrophage comprising a genetic disruption in the gene encoding iPLA2β in the engineered macrophage. In some embodiments, the iPLA2β gene disruption has been induced by CRISPR-Cas9. In some embodiments, the disruption comprises disrupting the iPLA2β gene at the DNA level, the disruption is not reversible, or the disruption is not transient. In some embodiments, the macrophage is a peritoneal macrophage.

The present disclosure also relates to a pharmaceutical composition comprising the engineered macrophage and a pharmaceutically-acceptable carrier, wherein the macrophage comprises a genetic disruption in the gene encoding iPLA2β.

The present disclosure also relates to a method of treating Type 1 Diabetes in a subject, the method comprising administering a therapeutically effective amount of a pharmaceutical composition comprising an engineered macrophage comprising a genetic disruption in the gene encoding iPLA2β in the engineered macrophage. In some embodiments, the iPLA2β gene disruption has been induced by CRISPR-Cas9. In some embodiments of the method, the disruption comprises disrupting the iPLA2β gene at the DNA level, the disruption is not reversible, or the disruption is not transient. In some embodiments, the engineered macrophage is a peritoneal macrophage. In some embodiments of the method, the pharmaceutical composition comprises a pharmaceutically-acceptable carrier. In some embodiments, the pharmaceutical composition is administered intra-arterially, intravenously, intrapleurally, intravesicularly, by peritoneal injection, or orally. In some embodiments, the pharmaceutical composition is administered at two or more time points, wherein the time points are separated by at least 24 hours. In some embodiments, the subject is at risk of developing Type 1 Diabetes or exhibits symptoms of Type 1 Diabetes.

### BRIEF DESCRIPTION OF THE DRAWINGS

The present invention will now be described by way of example, with reference to the accompanying drawings, in which:

FIGS. 1A-1M show the effects of temporal FKGK18 regimen on T1D incidence and islet phenotype, wherein female NOD mice were administered FKGK18 (20 mg/kg, 3 × weekly) or vehicle (PBS-T) starting at 4 or 8 weeks of age. FIGS. 1A and 1B show diabetes incidence; blood glucose was monitored weekly in the 4-week (FIG. 1A; n = 17 and 15 for PBS-T and FKGK18 groups, respectively) and 8-week (FIG. 1B; n = 15 each in the PBS-T and FKGK18 groups) regimen groups for up to 30 weeks. Two consecutive readings of ≥ 275 mg/dL were recorded as onset of T1D (^{†}P < 0.05). FIGS. 1C-1F show glucose tolerance test (GTT) results, wherein overnight fasted mice were administered glucose (2 g/kg, i.p.), glucose levels in blood from tail vein were monitored over a 2-hour period, and AUC were generated. FIGS. 1C and 1D show four-week group at 14 weeks of age; n = 5 each in the PBS-T and FKGK18 groups. FIGS. 1E and 1F show eight-week group at 25 weeks of age; n = 7 and 5 for PBS-T and FKGK18 groups, respectively. FIGS. 1G-1I show phenotype parameters in the 8-week regimen group. FIG. 1G shows urinary PGE₂ metabolites (PGEMs, n = 6 in each group, 18 weeks of age). FIGS. 1H and 1I show β Cell mass (PBS-T, n = 15; FKGK18, n = 14) (FIG. 1H) and circulating insulin (n = 15 in each group) (FIG. 1I) were determined at sacrifice (PBS-T, 14-30 weeks of age; FKGK18, 16-36 weeks of age). FIGS. 1J and 1K show islet infiltration; paraffin sections (10 µm) of pancreas were prepared and stained with H&E. Percent infiltration for each islet was calculated as the value of noninfiltrated area subtracted from total islet area (% infiltrate = 100 × [(total area - noninfiltrated area)/(total area)]) using ImageJ software. (PBS-T, n = 14 and 166 islets; FKGK18, n = 15 and 260 islets). FIG. 1J shows islet infiltration range. FIG. 1K shows average islet infiltration. FIGS. 1L and 1M show islet immune cell phenotype; paraffin sections (10 µm) of pancreas were prepared and stained for CD4+-T cells or B (B220) cells. Data presented are mean ± SEM of CD4+ T cells or B cells per islet. FIG. 1L shows quantitation of CD4± T cells per islet (PBS-T, n = 14 and 223 islets; FKGK18, n = 15 and 290 islets). FIG. 1M shows quantitation of B cells per islet (PBS-T, n = 14 and 213 islets; FKGK18, n = 15 and 328 islets). Statistical analyses: (FIGS. 1A and 1B) Mantel-Cox test; (FIGS. 1D-1M) Student’s t test.

FIGS. 2A-2E show the effects of FKGK18-withdrawal regimen on T1D incidence and glucose tolerance, wherein female NOD mice were administered FKGK18 (20 mg/kg, 3 × weekly, n = 18) or vehicle (PBS-T, n = 17) starting at 10 days of age and until 14 weeks of age. FIG. 2A shows T1D incidence, wherein blood glucose was monitored weekly for up to 30 weeks, and 2 consecutive readings of ≥ 275 mg/dL were recorded as onset of T1D. FIGS. 2B and 2D show glucose tolerance test (GTT) results, assessed at 14 (FIG. 2B) and 25 (FIG. 2D) weeks of age (data are presented as mean ± SEM), as described in FIG. 1. FIGS. 2C and 2E show corresponding AUC. N values for PBS-T & FKGK18: 8 and 8 (FIGS. 2B and 2C); 8 and 6 (FIGS. 2D and 2E), respectively. Statistical analyses: (FIG. 2A) Mantel-Cox test; (FIGS. 2C and 2E) Student’s t test.

FIGS. 3A-3E show NOD.iPLA2β^{+/-} genotype and diabetes phenotype. FIG. 3A shows genotype. DNA was generated from tail clips and progeny were genotyped by PCR analyses. Reactions were performed in the presence of primers for the WT sequence (NOD) or for the disrupted sequence (NOD-HET) for each mouse. The expected bands for the WT (1400 bp) and HET (1400 and 400 bp) in 2 mice each are presented. L, bp ladder. FIG. 3B shows T1D incidence, wherein blood glucose was monitored weekly for up to 30 weeks, and 2 consecutive readings of ≥ 275 mg/dL were recorded as onset of diabetes (n = 12 and 17 for NOD and NOD-HET groups, respectively). NOD-HET significantly different from NOD; ^{¥}P < 0.001. FIG. 3C shows RNA was isolated from NOD (n = 3) and NOD-HET (n = 3) macrophages and cDNA prepared for iPLA2β mRNA analyses by qPCR. FIG. 3D shows production of TNF-α by CD4+ T cells, wherein splenocytes were prepared from the NOD and NOD-HET, and CD4+ T cells were isolated and activated. The media was collected at 72 hours, and TNF-α concentration was determined by ELISA (n = 3 per group). FIG. 3E shows RNA was isolated from NOD (n = 3) and NOD-HET (n = 3) macrophages and cDNA prepared for Arg1. Statistical analyses: (FIG. 3B) Mantel-Cox test; (FIGS. 3C-3E) Student’s t test.

FIGS. 4A-4J show a comparison of eicosanoid production by Mϕ_{NOD} and Mϕ_{NOD-HET}. Peritoneal MΦ isolated from female NOD and NOD-HET mice were treated with vehicle control (DMSO) or classically activated with IFN-y + LPS, and the media was collected for eicosanoid analyses at 16 hours. The data (estimated marginal mean ± SEM) represent fold-change in activated lipids, relative to corresponding control. Mϕ_{NOD} (n = 9 and 5) and Mϕ_{NOD-HET} (n = 4 and 3) at 4 and 8 weeks, respectively. Levels of 6-Keto PGF₁α (FIG. 4A), 8-Iso PGF₂α (FIG. 4B), PGE₂ (FIG. 4C), PGA₂ (FIG. 4D), Proinflammatory prostaglandin (PG) pool (FIG. 4E), 20-HETE (FIG. 4F), 5-HETE (FIG. 4G), and PGE₁ (FIG. 4H) are shown. Proinflammatory (FIG. 4I) and antiinflammatory PGE₁ (FIG. 4J) are shown at 14 weeks. NOD-HET significantly different from NOD, ^{†}P < 0.05; ^{δ}P < 0.01; ^{#}P < 0.005; ^{¥}P < 0.001, n = 9 in each group. Statistical analyses: (FIGS. 4A-4H) multivariate 2-way ANOVA and time-course ANOVA; (FIG. 4I and4 J) Student’s t test.

FIGS. 5A-5I show a comparison of select plasma lipids during the prediabetic phase. Plasma was prepared from NOD (n = 5) and NOD-HET (n = 5) and processed for lipidomics analyses of eicosanoids (FIGS. 5A-5C), fatty acids (FIG. 5D), and sphingolipids (FIGS. 5E-5I). The data (mean ± SEM) represent pmol of each lipid species in 100 µL (FIGS. 5A-5D) or 50 µL (FIGS. 5E-5I) plasma. Levels of DHETs (FIG. 5A), Leukotrienes (FIG. 5B), EETs (FIG. 5C), EPA and DHGLA (FIG. 5D), Sphingosine and sphinganine (phosphorylated/nonphosphorylated) (FIG. 5E), Ceramides (FIG. 5F), Monohexosyl Ceramides (FIG. 5G), Sphingomyelins (FIG. 5H), and Ceramide-1-phosphates (FIG. 5I) are shown. NOD-HET significantly different from NOD, ^{†}P < 0.05; ^{δ}P < 0.01; ^{#}P < 0.005; ^{π}P < 0.0005. Statistical analyses: Student’s t test. UD, undetected.

FIGS. 6A-6I show a comparison of select plasma lipids at T1D onset. NOD mice were treated with PBS-T or with FKGK18, starting at 10 days of age, and sacrificed at the onset of T1D (d) or at 30 weeks if they remained nondiabetic (nd). Plasma was prepared from these mice and processed for lipidomics analyses. The data (mean ± SEM) represent pmol of each lipid species in 100 or 50 µL plasma. Levels of LTC₄ (FIG. 6A), 15-HETE (FIG. 6B), 5-HETE (FIG. 6C), PGD₂ (FIG. 6D), AA (FIG. 6E), So1P/So (FIG. 6F), EET/DHET (FIG. 6G), Resolvin D2 (FIG. 6H), and DHA (FIG. 6I) are shown. n = 3, 4, and 4 for PBS-T (P [nd]), PBS-T (P [d]), and FKGK18 (FK [nd]), respectively. P (d) significantly different from the other groups, ^{†}P < 0.05; ^{δ}P < 0.01; ^{Δ}P < 0.000001. One-way ANOVA.

FIGS. 7A-7G show diabetic and nondiabetic human plasma lipidome. Lipidomics analyses were performed in plasma from euglycemic autoantibody negative (Aab⁻), 1 Aab-positive (Aab⁺), and 2 Aab-positive (Aab⁺⁺), and recent T1D onset (3.34 ± 0.24 months T1D duration) (RO) subjects. The number of subjects, sex (female [F]/male [M]) distribution, and age (years) at visit are: Aab⁻, 10, 2F/8M, 9.26 ± 1.68; Aab⁺, 11, 6F/5M, 14.60 ± 1.38; Aab⁺⁺, 11, 8F/3M, 12.43 ± 1.66; RO, 13, 9F/4M, 8.99 ± 1.33. FIGS. 7A-7F show fold-abundances in lipids, relative to Aab⁻, are presented with mean ± SEM. FIG. 7G shows blood glucose at sample collection. Statistical analyses: (FIGS. 7A-7F) Pearson, Kendall, and Spearman’s rank order correlation; FIG. 7G, Student’s t test. All n is the same as previous panels, except RO = 12.

FIGS. 8A-8H show sphingolipids production by Mϕ_{NOD} and Mϕ_{C57}, in particular basal ceramides (FIG. 8A), activated ceramides (FIG. 8B), basal monohexyl ceramides (FIG. 8C), activated monohexyl ceramides (FIG. 8D), basal sphingomyelins (FIG. 8E), activated sphingomyelins (FIG. 8F), basal ceramide-1-phosphates (FIG. 8G), and activated ceramide-1-phosphates (FIG. 8H), wherein peritoneal macrophages isolated from female NOD (n=4) and age-matched C57BL/J6 from Jackson Laboratories (n=4) were treated with vehicle (DMSO, basal) alone or classically activated with IFNy+LPS and the cells collected for sphingolipids analyses. The data (mean±SEMs) are pmol lipid/10⁶ cells. (Statistical analyses: Students′ t-test. Mϕ_{NOD} significantly different from Mϕ_{C57}, ^{†}p<0.05; ^{∂}p<0.01; ^{#}p<0.005; ^{¥}p<0.001, *p<0.0001.).

FIGS. 9A-9K show a comparison of eicosanoid production by Mϕ_{NOD} and Mϕ_{NOD-HET}. Peritoneal macrophages (Mϕ) isolated from female NOD and NOD-HET mice were treated with vehicle control (DMSO) or classically activated with IFNy+LPS and the media collected for eicosanoid analyses at 16h. The data are means±SEMs of fold-change with activation, relative to corresponding controls. Mϕ_{NOD} (n = 9 & 5) and Mϕ_{NOD-HET} (n = 4 & 3) at 4 & 8 weeks, respectively. Fold-change is shown for 6-Keto PGF₂α (FIG. 9A), 8-IsoPGF₂α (FIG. 9B), PGE₂ (FIG. 9C), PGA₂ (FIG. 9D), pro-inflammatory pool (FIG. 9E), 20-HETE (FIG. 9F), 5-HETE (FIG. 9G), PGE₁ (FIG. 9H), proinflammatory lipids (FIG. 9I), proinflammatory PGE₂ and PGA₂ (FIG. 9J), and anti-inflammatory PGE₁ (FIG. 9K) at 14 week. (n=9 in each group.).

FIG. 10 shows blood glucose monitoring in NOD mice. The data (mean±SEM) are weekly blood glucose measurements in female NOD mice between 8 and 30 weeks of age. The data are pooled measurements from multiple cohorts (n=42) utilized in the various studies. The blood glucose values recorded at the onset of diabetes (≥275 mg/dl) were carried through till the end of the study for the purpose of generating this figure. Lipidomics analyses were performed with mice at ages (arrows) that were not associated with hyperglycemia.

FIGS. 11A-11I show a comparison of select plasma lipids in diabetic NOD mice. NOD mice were treated with PBS-Tor with FKGK18, starting at 10 days or 4 weeks of age, and sacrificed at the onset of diabetes. Plasma was prepared from these mice and processed for lipidomics analyses. The data (mean±SEM) represent pmol of each lipid species in 100 or 50 µL plasma fo LTC₄ (FIG. 11A), 15-HETE (FIG. 11B), 5-HETE (FIG. 11C), PGD₂ (FIG. 11D), AA (FIG. 11E), So1P/So (FIG. 11F), EET/DHET (FIG. 11G), resolving D2 (FIG. 11H), and DHA (FIG. 11I). (Statistical analyses: Students′ t-test. N=4/group.).

FIGS. 12A and 12B show macrophage adoptive transfer. Peritoneal macrophages were obtained from 8 week old female NOD and NOD.iPLA2β^{-/-} (NOD-KO). Female NOD mice (at 8 weeks of age) were administered the macrophages (i.p., 2.75×10⁶) from NOD (n=11) or NOD-KO (n=14). FIG. 12A shows iPLA2β mRNA; macrophage phenotype was verified by RT-qPCR. FIG. 12B shows diabetes incidence, wherein blood glucose was monitored weekly for up to 30 weeks. Two consecutive readings of ≥275 mg/dL were recorded as onset of T1D. (Statistical analyses: Mantel-Coxtest.) UD, undetected.

### DETAILED DESCRIPTION

Provided are cells for adoptive cell therapy, e.g., adoptive immunotherapy. The cells include macrophages in which the iPLA₂β gene is disrupted. Also provided are methods and uses of the cells, such as in adoptive therapy in the treatment or prevention of type 1 diabetes (T1D). Also provided are methods for engineering, preparing, and producing the cells, and compositions containing the cells. In some aspects, the provided embodiments provide an improved composition and method for the treatment of T1D. Among the cells disclosed herein are those in which certain genes and/or gene products have been disrupted, modified and/or repressed, in particular via disruption that impairs or reduces expression of iPLA₂β.

As used herein, “repression” of gene expression refers to the elimination or reduction of expression of one or more gene products encoded by the subject gene in a cell, compared to the level of expression of the gene product in the absence of the repression. Exemplary gene products include mRNA and protein products encoded by the gene. Repression in some cases is transient or reversible and in other cases is permanent. Repression in some cases is of a functional or full-length protein or mRNA, despite the fact that a truncated or non-functional product may be produced. In some embodiments herein, gene activity or function, as opposed to expression, is repressed. Gene repression is generally induced by artificial methods, i.e., by addition or introduction of a compound, molecule, complex, or composition, and/or by disruption of nucleic acid of or associated with the gene, such as at the DNA level. Exemplary methods for gene repression include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination.

As used herein, a “disruption” of a gene refers to a change in the sequence of the gene, at the DNA level. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

As used herein, the singular forms “a,” “an,” and “the” include plural referents unless the context clearly dictates otherwise. For example, “a” or “an” means “at least one” or “one or more.”

Various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a subject includes any living organism, such as humans and other mammals. Mammals include, but are not limited to, humans, and non-human animals, including farm animals, sport animals, rodents and pets. In some embodiments, the subject is at risk of developing Type 1 Diabetes. A subject may be at risk of developing Type 1 Diabetes for a number of reasons, such as the presence of certain genetic risk loci and other factors known to those of skill in the art.

As used herein, the terms “treatment,” “treat,” and “treating,” refer to complete or partial amelioration or reduction of a disease or condition or disorder, or a symptom, adverse effect or outcome, or phenotype associated therewith. In certain embodiments, the effect is therapeutic, such that it partially or completely cures a disease or condition or adverse symptom attributable thereto. In certain embodiments, the effective is preventative.

As used herein, a “therapeutically effective amount” of a compound or composition or combination refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

Disclosed herein are methods for the treatment or prevention of Type 1 Diabetes through administration of macrophages to a subject, wherein the expression of iPLA₂β in the macrophages is disrupted. Such macrophages exhibit less inflammation than macrophages in which iPLA₂β is not disrupted and can delay or prevent the onset of Type 1 Diabetes in the subject. The disruption of iPLA₂β in the macrophages can be performed ex vivo prior to administration to a subject. The disruption may be performed using CRISPR-Cas9 (see, e.g., Chen et al. Not Commun. 2021 Jun 15;12(1):3644).

In some aspects, the disruption is carried out by gene editing, such as using a DNA binding protein or DNA-binding nucleic acid, which specifically binds to or hybridizes to the gene at a region targeted for disruption. In some aspects, the protein or nucleic acid is coupled to or complexed with a nuclease, such as in a chimeric or fusion protein. For example, in some embodiments, the disruption is effected using a fusion comprising a DNA-targeting protein and a nuclease, such as a Zinc Finger Nuclease (ZFN) or TAL-effector nuclease (TALEN), or an RNA-guided nuclease such as a clustered regularly interspersed short palindromic nucleic acid (CRISPR)-Cas system, such as CRISPR-Cas9 system, specific for the gene being disrupted.

The cells and compositions containing the cells for engineering typically are isolated from a sample, such as a biological sample, e.g., one obtained from or derived from a subject. In some embodiments, the subject from which the cell is isolated as one having a particular disease or condition or in need of a cell therapy or to which cell therapy will be administered. The subject in some embodiments is a mammal, such as a human, such as a subject in need of a particular therapeutic intervention, such as the adoptive cell therapy for which cells are being isolated, processed, and/or engineered.

In some embodiments, gene repression is carried out using one or more DNA-binding nucleic acids, such as disruption via an RNA-guided endonuclease (RGEN), or other form of repression by another RNA-guided effector molecule. For example, in some embodiments, the repression is carried out using clustered regularly interspaced short palindromic repeats (CRISPR) and CRISPR-associated (Cas) proteins. See Sander and Joung, Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some embodiments, the CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains).

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes or Staphylococcus aureus.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, the CRISPR system induces DSBs at the target site, followed by disruptions as discussed herein. In other embodiments, Cas9 variants, deemed “nickases” are used to nick a single strand at the target site. In some aspects, paired nickases are used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

In general, a CRISPR system is characterized by elements that promote the formation of a CRISPR complex at the site of a target sequence. Typically, the In the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. In some embodiments, the target sequence is located in the nucleus or cytoplasm of the cell. In some embodiments, the target sequence may be within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of the CRISPR complex.

As with the target sequence, in some embodiments, complete complementarity is not necessarily needed. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of the CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to the cell.

In some embodiments, a vector comprises a regulatory element operably linked to an enzyme-coding sequence encoding the CRISPR enzyme, such as a Cas protein. Non-limiting examples of Cas proteins include Cas 1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csxl, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes, S. aureus or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, a vector encodes a CRISPR enzyme that is mutated to with respect to a corresponding wild-type enzyme. Non-limiting examples of mutations in a Cas9 protein are known in the art (see e.g. WO2015/161276), any of which can be included in a CRISPR/Cas9 system in accord with the provided methods. In some embodiments, the CRISPR enzyme is mutated such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. In some embodiments, one or more codons (e.g. 1, 2, 3, 4, 5, 10, 15, 20, 25, 50, or more, or all codons) in a sequence encoding the CRISPR enzyme corresponds to the most frequently used codon for a particular amino acid.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to the cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide sequence reactions.

A guide sequence may be selected to target any target sequence. In some embodiments, the target sequence is a sequence within a genome of a cell. Exemplary target sequences include those that are unique in the target genome. In some embodiments, a guide sequence is selected to reduce the degree of secondary structure within the guide sequence. Secondary structure may be determined by any suitable polynucleotide folding algorithm.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences.

Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In some aspects, loop forming sequences for use in hairpin structures are four nucleotides in length, and have the sequence GAAA. However, longer or shorter loop sequences may be used, as may alternative sequences. In some embodiments, the sequences include a nucleotide triplet (for example, AAA), and an additional nucleotide (for example C or G). Examples of loop forming sequences include CAAA and AAAG. In some embodiments, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence, such as a polyT sequence, for example six T nucleotides.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CR ISPR enzyme are described in US20110059502, incorporated herein by reference. In some embodiments, a tagged CRISPR enzyme is used to identify the location of a target sequence.

In some embodiments, a CRISPR enzyme in combination with (and optionally complexed with) a guide sequence is delivered to the cell. In some embodiments, methods for introducing a protein component into a cell according to the present disclosure (e.g. Cas9/gRNA RNPs) may be via physical delivery methods (e.g. electroporation, particle gun, Calcium Phosphate transfection, cell compression or squeezing), liposomes or nanoparticles.

For example, CRISPR/Cas9 technology may be used to knock-down gene expression of the target antigen in the engineered cells. In an exemplary method, Cas9 nuclease (e.g., that encoded by mRNA from Staphylococcus aureus or from Stretpococcus pyogenes, e.g. pCW-Cas9, Addgene #50661, Wang et al. (2014) Science, 3:343-80-4; or nuclease or nickase lentiviral vectors available from Applied Biological Materials (ABM; Canada) as Cat. No. K002, K003, K005 or K006) and a guide RNA specific to the target antigen gene are introduced into cells, for example, using lentiviral delivery vectors or any of a number of known delivery method or vehicle for transfer to cells, such as any of a number of known methods or vehicles for delivering Cas9 molecules and guide RNAs. Degree of Knockout of a gene (e.g., 24 to 72 hours after transfer) is assessed using any of a number of well-known assays for assessing gene disruption in cells.

It is within the level of a skilled artisan to design or identify a gRNA sequence that is or comprises a sequence targeting a target antigen of interest, such as any described herein, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; http://www.e-crisp.org/E-CRISP/). In some embodiments, the gRNA sequence is or comprises a sequence with minimal off-target binding to a non-target gene.

In some embodiments, design gRNA guide sequences and/or vectors for any of the antigens as described herein are generated using any of a number of known methods, such as those for use in gene knockdown via CRISPR-mediated, TALEN-mediated and/or related methods.

In some aspects, target polynucleotides are modified in a eukaryotic cell. In some embodiments, the method comprises allowing the CRISPR complex to bind to the target polynucleotide to effect cleavage of said target polynucleotide thereby modifying the target polynucleotide, wherein the CRISPR complex comprises the CRISPR enzyme complexed with a guide sequence hybridized to a target sequence within said target polynucleotide, wherein said guide sequence is linked to a tracr mate sequence which in turn hybridizes to a tracr sequence.

In some aspects, the methods include modifying expression of a polynucleotide in a eukaryotic cell. In some embodiments, the method comprises allowing the CRISPR complex to bind to the polynucleotide such that said binding results in increased or decreased expression of said polynucleotide; wherein the CRISPR complex comprises a CRISPR enzyme complexed with a guide sequence hybridized to a target sequence within said polynucleotide, wherein said guide sequence is linked to a tracr mate sequence which in turn hybridizes to a tracr sequence.

In some embodiments, a CRISPR/Cas system can be used for knocking down, such as reducing or suppressing, the expression of a target sequence. Exemplary features of CRISPR/Cas systems are described below and can be adapted for use in reducing or suppressing expression of a molecule, rather than disrupting or deleting a gene encoding the molecule, by using an enzymatically inactive nuclease. In some embodiments, a guide RNA (gRNA) targeting a gene of interest, such as any described herein, or the promoter, enhancer or other cis- or trans-acting regulatory regions associated therewith, can be introduced in combination with a modified Cas9 protein or a fusion protein containing the modified Cas9 protein, to suppress the expression of, e.g., knock-down, of the gene(s). In some embodiments, the Cas9 molecule is an enzymatically inactive Cas9 (eiCas9) molecule, which comprises a mutation, e.g., a point mutation, that causes the Cas9 molecule to be inactive, e.g., a mutation that eliminates or substantially reduces the Cas9 molecule cleavage activity (see e.g. WO2015/161276). In some embodiments, the eiCas9 molecule is fused, directly or indirectly to, a transcription activator or repressor protein.

Macrophages can be obtained from a subject by means known to those of skill in the art including, but not limited to, isolation from peripheral blood of the subject. In some embodiments, the macrophages administered to a subject are autologous. In some embodiments, the macrophages administered to a subject are allogeneic.

In some embodiments, the cells and cell populations are administered to a subject in the form of a composition, such as a pharmaceutical composition. In some embodiments, the pharmaceutical composition further comprises other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cell populations are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

In some aspects, the choice of carrier can in the pharmaceutical composition is determined in part by the particular macrophages, as well as by the particular method used to administer the macrophages. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known to those of skill in the art.

In certain embodiments, a pharmaceutical composition comprising a macrophage population described herein can be formulated as an inclusion complex, such as cyclodextrin inclusion complex, or as a liposome. Liposomes can serve to target the macrophages to a particular tissue. Many methods are available for preparing liposomes, such as those described in, for example, U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician.

The pharmaceutical composition in some embodiments comprises the cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges. In certain embodiments, fewer than one million cells are administered.

The cells and compositions in some embodiments are administered using standard administration techniques, formulations, and/or devices. Provided are formulations and devices, such as syringes and vials, for storage and administration of the compositions. Administration can be autologous or heterologous. For example, immunoresponsive cells or progenitors can be obtained from one subject, and administered to the same subject or a different, compatible subject. Peripheral blood derived immunoresponsive cells of the invention or their progeny (e.g., in vivo, ex vivo or in vitro derived) can be administered via localized injection, including catheter administration, systemic injection, localized injection, intravenous injection, or parenteral administration. When administering a therapeutic composition of the present invention (e.g., a pharmaceutical composition containing a genetically modified macrophage), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

Compositions of the cells in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the genetically engineered in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cell populations are administered prior to the one or more additional therapeutic agents. In some embodiments, the cell populations are administered after to the one or more additional therapeutic agents.

Once the cells are administered to a mammal (e.g., a human), the biological activity of the engineered cell populations in some aspects is measured by any of a number of known methods. Parameters to assess include, but are not limited to, assessment of the lymphocyte repertoire in response to macrophages in vivo, e.g., by imaging, or ex vivo, e.g., by ELISA or flow cytometry. In certain embodiments, the biological activity of the cells also can be measured by assaying expression and/or secretion of certain cytokines, such as IFNy, IL-2, and TNF. In some aspects the biological activity is measured by assessing clinical outcome, such as reduction in blood glucose or a prevention of T1D onset.

Although illustrative aspects of the invention have been disclosed in detail herein, the invention is not limited to those precise aspects. Various changes and modifications can be effected by one skilled in the art without departing from the scope of the invention as defined by the appended claims and their equivalents.

### EXAMPLES

### Nomenclature

Mice described herein include spontaneous diabetes-resistant C57BL/6J, spontaneous diabetes-prone NOD, and NOD.PLA2G6^{+/-}. These strains are designated C57, NOD, and NOD-HET, respectively. MΦ from these are designated Mϕ_{C57}, Mϕ_{NOD}, and Mϕ_{NOD-HET}, respectively.

### Age-Dependent Impact of iPLA2β Inhibition on T1D Development

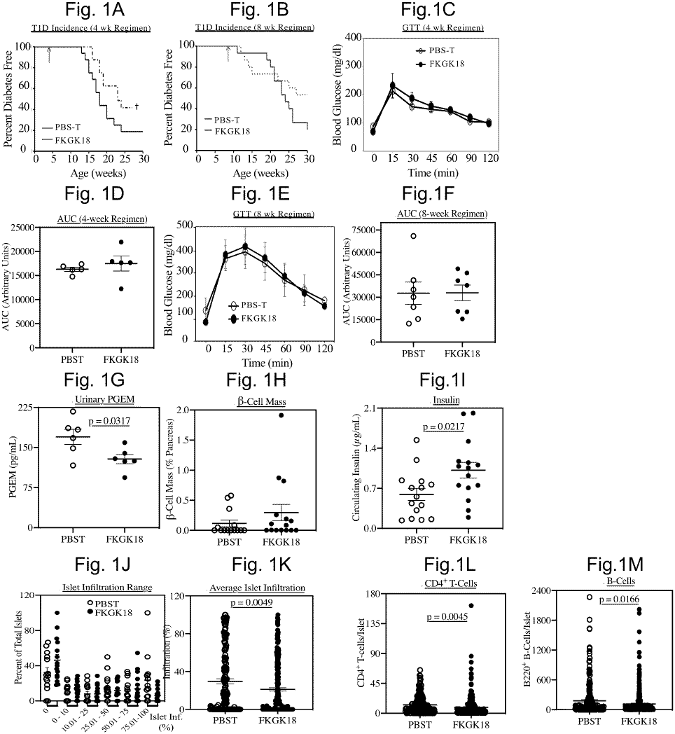

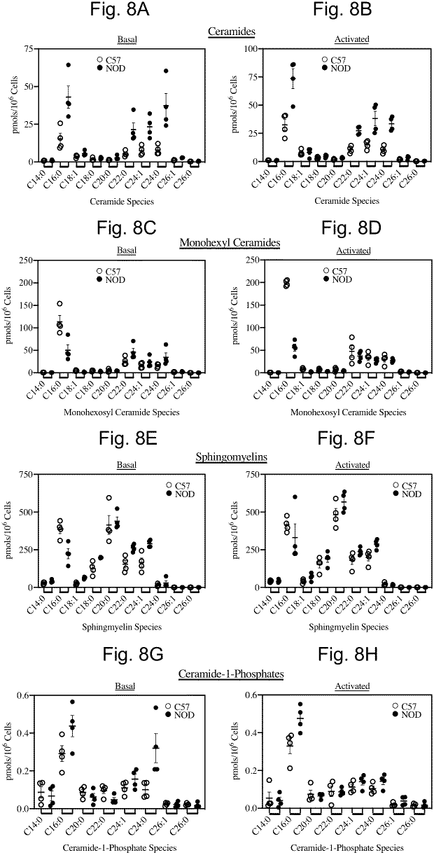

As the female NOD exhibit a recognized progression in T1D development, where onset of insulitis commences at about 4 weeks of age and the inflammatory processes ramp up at about 8 weeks of age, Applicants monitored T1D development in female NOD administered FKGK18 starting at 10 days and 4 or 8 weeks of age. 80%-90% of vehicle-treated NOD became diabetic by 25-30 weeks of age in the 10-day group, but only 10%-15% NOD administered FKGK18 developed T1D (data not shown). The vehicle-treated (PBS-T-treated) groups in the 4-week (FIG. 1A) and 8-week (FIG. 1B) groups also exhibited an 80% T1D incidence by 25-30 weeks of age. In contrast, 40% of mice in the 4-week FKGK18 group remained diabetes free (FIG. 1A). While there was evidence of a modest delay in T1D incidence in the 8-week FKGK18 group (FIG. 1B), it was not significantly different from the corresponding PBS-T group. No differences in glucose tolerance were noted between the groups started on PBS-T and FKGK18 at either 4 weeks (FIGS. 1C and 1D) or 8 weeks of age (FIGS. 1E and 1F).

Because the 8-week FKGK18-treated group appeared to be at the cusp of effective iPLA₂β intervention, Applicants further probed β cell and islet immune cell phenotype in this group. FKGK18 administration reduced urinary PGE₂ metabolites (PGEM, FIG. 1G), relative to the vehicle-treated mice, reflecting in vivo FKGK18-mediated inhibition of iPLA₂β activity. This was accompanied by similar β cell mass (FIG. 1H), higher circulating insulin (FIG. 1I), and reduced islet infiltration (FIGS. 1J and 1K). Furthermore, pancreatic islet abundances of CD4⁺ T cells (FIG. 1L) and B cells (FIG. 1M) were significantly reduced in the FKGK18-treated mice, relative to the PBS-T group. These findings reveal an age-dependent impact of iPLA₂β inhibition on T1D, with early intervention being more beneficial.

### Protective Effects of iPLA2β Inhibition Are Lost Upon FKGK18 Withdrawal

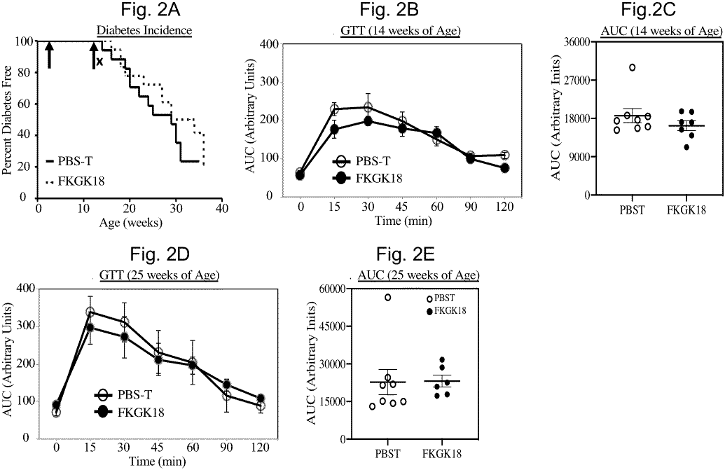

To determine if the protective effects of early intervention persist following inhibitor withdrawal, a concurrent cohort NOD group administered FKGK18 from 10 days until 14 weeks of age, an age closely associated with onset of T1D, was monitored for up to 30 weeks. Applicants determined that the decreased incidence in the NOD treated with FKGK18 continuously from 10 days until 30 weeks of age was not evident when FKGK18 was withdrawn after 14 weeks (FIG. 2A). Glucose tolerance was also indistinguishable between the PBS-T- and FKGK18-withdrawn groups (FIGS. 2B-2E). Taken together, these findings suggest that the protective effects of the reversible inhibitor FKGK18 are lost upon withdrawal.

### MΦ_{NOD} Exhibit a Profound Inflammatory Lipid Profile

In view of the above observations suggesting a temporal impact of iPLA₂β-derived lipids (iDL) on T1D development, Applicants examined the lipid profile in the NOD, as compared with C57. Mϕ are key to the autoimmune-mediated destruction of β cells, leading to T1D, as they are among the first cells to infiltrate the islets and trigger processes that promote infiltration of other immune cells. Our earlier assessments of specific phenotypic markers revealed that iPLA₂β activation promotes Mϕ polarization toward M1, whereas iPLA₂β deficiency favors M2 antiinflammatory polarization. Applicants targeted the MΦ lipid profile for analyses in the studies here. Peritoneal Mϕ were isolated from mice and treated with either vehicle control (DMSO) or activated with IFN-y + LPS. The media was collected for lipidomics analyses of eicosanoids, specialized proresolving mediators (SPMs), and fatty acids and the cells for sphingolipids. Multiple reaction monitoring (MRM) transitions with corresponding declustering potentials, collision energies, entrance potentials, and collision cell exit potentials are shown in Tables 1 and 2.

TABLE 1

| Analyte ID | Q1 Mass (Da) | Q2 Mass (Da) | DP (volt) | EP (volt) | CE (volt) | CXP (volt) |
|---|---|---|---|---|---|---|
| 6-keto PGF₁α-d4 | 373.2 | 167 | -80 | -14 | -33 | -15 |
| 6-keto PGF₁α | 369.2 | 163 | -80 | -14 | -33 | -15 |
| 8-iso PGF₂α-d4 | 357.4 | 197.1 | -150 | -13 | -33 | -14 |
| 8-iso PGF₂α | 353.4 | 193 | -130 | -13 | -31 | -11 |
| TXB₂-d4 | 373.2 | 173 | -80 | -13 | -23 | -15 |
| TXB₂ | 369.2 | 169 | -80 | -13 | -25 | -15 |
| 5-iPF₂α-VI-d11 | 364.2 | 115 | -90 | -12 | -28 | -20 |
| 5-iPF₂α-VI | 353.2 | 114.9 | -90 | -12 | -28 | -16 |
| PGE₂-d9 | 360.2 | 280.3 | -80 | -13 | -24 | -12 |
| PGE₂ | 351.2 | 271.2 | -80 | -13 | -25 | -12 |
| PGF₂α-d9 | 362.2 | 193 | -70 | -10 | -35 | -18 |
| PGF₂α | 353.2 | 193 | -70 | -10 | -31 | -18 |
| PGD₂-d9 | 360.201 | 280.3 | -80 | -10 | -24 | -12 |
| PGD₂ | 351.201 | 271.2 | -80 | -10 | -23 | -12 |
| RvD3-d5 | 380.22 | 147 | -68 | -10 | -24 | -11 |
| RvD3 | 375.22 | 147 | -68 | -10 | -24 | -11 |
| RvD2-d5 | 380.2 | 141 | -80 | -12 | -21 | -10 |
| RvD2 | 375.2 | 141 | -80 | -12 | -22 | -10 |
| PGE₁-d4 | 357.2 | 239 | -105 | -13 | -20 | -19 |
| PGE₁ | 353.2 | 235 | -105 | -13 | -19 | -19 |
| RvD1-d5 | 380.201 | 141 | -70 | -13 | -20 | -10 |
| RvD1 | 375.201 | 141 | -70 | -13 | -22 | -10 |
| Lipoxin A4-d5 | 356.3 | 114.9 | -90 | -14 | -21 | -20 |
| Lipoxin A4 | 351.3 | 114.8 | -90 | -13 | -20 | -15 |
| PGA₂-d4 | 337.2 | 275.3 | -80 | -14 | -19 | -12 |
| PGA₂ | 333.2 | 271.2 | -80 | -14 | -19 | -12 |
| LTD₄-d5 | 500.3 | 177 | -105 | -10 | -24 | -16 |
| LTD₄ | 495.3 | 176.9 | -105 | -10 | -19 | -14 |
| LTC₄-d5 | 629.3 | 272.1 | -50 | -13 | -30 | -13 |
| LTC₄ | 624.3 | 272.1 | -60 | -13 | -30 | -13 |
| LTE₄-d5 | 443.2 | 338.3 | -80 | -10 | -24 | -15 |
| LTE₄ | 438.2 | 333.1 | -80 | -10 | -23 | -15 |
| LTB₄-d4 | 339.2 | 197 | -95 | -14 | -21 | -16 |
| LTB₄ | 335.2 | 195 | -95 | -14 | -22 | -16 |
| maresin 2-d5 | 364.23 | 221.1 | -65 | -14 | -16 | -11 |
| maresin 2 | 359.23 | 221.1 | -65 | -14 | -16 | -11 |
| (±)14,15-DHET-d11 | 348.2 | 207 | -110 | -14 | -24 | -14 |
| (±)14,15-DHET | 337.2 | 207 | -110 | -14 | -26 | -14 |
| 15-deoxy-Δ12,14-PGJ₂-d4 | 319.2 | 275.3 | -100 | -9 | -22 | -10 |
| 15-deoxy-Δ12,14-PGJ₂ | 315.2 | 271.2 | -100 | -9 | -21 | -10 |
| (±)11,12-DHET-d11 | 348.2 | 167 | -85 | -12 | -25 | -14 |
| (±)11,12-DHET | 337.2 | 167 | -85 | -12 | -24 | -14 |
| (±)8,9-DHET-d11 | 348.2 | 185.2 | -93 | -9 | -23 | -13 |
| (±)8,9-DHET | 337.2 | 185.2 | -95 | -10 | -20 | -13 |
| 20-HETE-d6 | 325.2 | 281.3 | -85 | -13 | -21 | -12 |
| 20-HETE | 319.2 | 275.2 | -85 | -13 | -21 | -12 |
| 15 HETE-d8 | 327.2 | 226 | -116 | -13 | -16 | -16 |
| 15 HETE | 319.2 | 219 | -116 | -13 | -19 | -16 |
| 12 HETE-d8 | 327.2 | 184.1 | -90 | -13 | -20 | -16 |
| 12 HETE | 319.2 | 178.9 | -90 | -13 | -19 | -16 |
| (±)14(15)-EET-d11 | 330.2 | 219.1 | -90 | -13 | -15 | -15 |
| (±)14(15)-EET | 319.2 | 219.1 | -90 | -13 | -15 | -15 |
| 5 HETE d8 | 327.2 | 116 | -90 | -13 | -18 | -10 |
| 5 HETE | 319.2 | 115 | -90 | -13 | -20 | -10 |
| (±)8(9)-EET-d11 | 330.2 | 123 | -90 | -12 | -18 | -11 |
| (±)8(9)-EET | 319.2 | 123 | -90 | -12 | -18 | -11 |
| EPA d5 | 306.2 | 262.3 | -86 | -10 | -15 | -18 |
| EPA | 301.2 | 257.1 | -86 | -10 | -17 | -18 |
| DHA-d5 | 332.2 | 288.3 | -95 | -12 | -14 | -12 |
| DHA | 327.2 | 283.2 | -95 | -12 | -19 | -12 |
| AA-d8 | 311.2 | 267.3 | -150 | -13 | -18 | -16 |
| AA | 303.2 | 259.2 | -150 | -13 | -17 | -14 |
| DHGLA-d6 | 311.2 | 267.2 | -105 | -14 | -20 | -13 |
| DHGLA | 305.2 | 261.2 | -90 | -10 | -43 | -16 |

TABLE 2

| Analyte ID | Q1 Mass (Da) | Q2 Mass (Da) | DP (volt) | EP (volt) | CE (volt) | CXP (volt) |
|---|---|---|---|---|---|---|
| d17:1 So | 286.4 | 268.3 | 120 | 10 | 15 | 10 |
| d17:0 Sa | 288.4 | 270.4 | 120 | 10 | 21 | 10 |
| d18:1 So | 300.5 | 282.3 | 120 | 10 | 21 | 10 |
| d18:0 Sa | 302.5 | 284.3 | 120 | 10 | 21 | 10 |
| d17:1 So1P | 366.4 | 250.4 | 120 | 10 | 23 | 10 |
| d17:0 Sa1P | 368.4 | 252.4 | 120 | 10 | 23 | 10 |
| d18:1 So1P | 380.4 | 264.4 | 120 | 10 | 25 | 10 |
| d18:0 Sa1P | 382.4 | 266.4 | 120 | 10 | 25 | 10 |
| C12 Cer | 482.6 | 264.4 | 80 | 10 | 41 | 10 |
| C14 Cer | 510.7 | 264.4 | 80 | 10 | 43.5 | 10 |
| C16 Cer | 538.7 | 264.4 | 80 | 10 | 46 | 10 |
| C18:1 Cer | 564.7 | 264.4 | 80 | 10 | 48.5 | 10 |
| C18:0 Cer | 566.7 | 264.4 | 80 | 10 | 48.5 | 10 |
| C20 Cer | 594.7 | 264.4 | 80 | 10 | 51 | 10 |
| C22 Cer | 622.8 | 264.4 | 80 | 10 | 53.5 | 10 |
| C24:1 Cer | 648.9 | 264.4 | 80 | 10 | 56 | 10 |
| C24 Cer | 650.9 | 264.4 | 80 | 10 | 56 | 10 |
| C26:1 Cer | 676.9 | 264.4 | 80 | 10 | 58.5 | 10 |
| C26 Cer | 678.9 | 264.4 | 80 | 10 | 58.5 | 10 |
| C12 C1P | 562.4 | 264.4 | 80 | 10 | 41 | 10 |
| C14 C1P | 590.4 | 264.4 | 80 | 10 | 43.5 | 10 |
| C16 C1P | 618.5 | 264.4 | 80 | 10 | 46 | 10 |
| C18:1 C1P | 644.5 | 264.4 | 80 | 10 | 48.5 | 10 |
| C18:0 C1P | 646.5 | 264.4 | 80 | 10 | 48.5 | 10 |
| C20 C1P | 674.4 | 264.4 | 80 | 10 | 51 | 10 |
| C22 C1P | 702.7 | 264.4 | 80 | 10 | 53.5 | 10 |
| C24:1C1P | 728.6 | 264.4 | 80 | 10 | 56 | 10 |
| C24 C1P | 730.6 | 264.4 | 80 | 10 | 56 | 10 |
| C26:1 C1P | 756.7 | 264.4 | 80 | 10 | 58.5 | 10 |
| C26 C1P | 758.7 | 264.4 | 80 | 10 | 58.5 | 10 |
| C12 MonHex | 644.6 | 264.4 | 80 | 10 | 41 | 10 |
| C14 MonHex | 672.6 | 264.4 | 80 | 10 | 43.5 | 10 |
| C16 MonHex | 700.7 | 264.4 | 80 | 10 | 46 | 10 |
| C18:1 MonHex | 726.7 | 264.4 | 80 | 10 | 48.5 | 10 |
| C18:0 MonHex | 728.7 | 264.4 | 80 | 10 | 48.5 | 10 |
| C20 MonHex | 756.7 | 264.4 | 80 | 10 | 51 | 10 |
| C22 MonHex | 784.8 | 264.4 | 80 | 10 | 53.5 | 10 |
| C24:1 MonHex | 810.9 | 264.4 | 80 | 10 | 56 | 10 |
| C24 MonHex | 812.9 | 264.4 | 80 | 10 | 56 | 10 |
| C26:1 MonHex | 838.9 | 264.4 | 80 | 10 | 58.5 | 10 |
| C26 MonHex | 840.9 | 264.4 | 80 | 10 | 58.5 | 10 |
| C12 SM | 647.7 | 184.4 | 80 | 10 | 41 | 10 |
| C14 SM | 675.7 | 184.4 | 80 | 10 | 43.5 | 10 |
| C16 SM | 703.8 | 184.4 | 80 | 10 | 46 | 10 |
| C18:1 SM | 729.8 | 184.4 | 80 | 10 | 48.5 | 10 |
| C18:0 SM | 731.8 | 184.4 | 80 | 10 | 48.5 | 10 |
| C20 SM | 759.9 | 184.4 | 80 | 10 | 51 | 10 |
| C22 SM | 787.9 | 184.4 | 80 | 10 | 53.5 | 10 |
| C24:1 SM | 813.9 | 184.4 | 80 | 10 | 56 | 10 |
| C24 SM | 815.9 | 184.4 | 80 | 10 | 56 | 10 |
| C26:1 SM | 841.9 | 184.4 | 80 | 10 | 58.5 | 10 |
| C26 SM | 843.9 | 184.4 | 80 | 10 | 58.5 | 10 |

Eicosanoids and fatty acids. Metabolites of arachidonic acid are recognized to be pro- or antiinflammatory. In comparison with Mϕ_{C57}, production of several proinflammatory prostaglandins (PGs) by MΦ_{NOD} was significantly higher under both basal and activated conditions (Table 3A). The most profoundly affected lipids included 6-keto PGF₁α, 8-Iso PGF₂α, 5-IPFα-VI, PGE₂, PGA₂, and 15-deoxy-Δ12,14-PGJ₂. Furthermore, LT (LTD₄, LTC₄, and LTE₄) production by Mϕ_{C57} was significantly increased under basal conditions, and LTD₄ production remained higher under activating conditions, in comparison with production by M_{C57}. Production of HETEs, DHETs, or PGE₁ was not significantly different between the 2 groups under basal conditions, but under activating conditions, production of 12-HETE, (±) 8,9-DHET, and PGE₁ by Mϕ_{NOD} was significantly higher, relative to Mϕ_{C57} (Tables 3 and 4). Cellular lipidomic analyses identified several SPMs, including resolvin D2 and D1 (from docosahexaenoic acid [DHA]), and lipoxin A4 (from arachidonic acid [AA]). However, production of these or fatty acids EPA, DHA, and AA (Table 5) by Mϕ_{NOD} and Mϕ_{C57} was not different under basal or activated conditions. Table 3 shows proinflammatory eicosanoids production by Mϕ_{NOD}, relative to Mϕ_{C57}. Table 4 shows anti-inflammatory eicosanoids production by Mϕ_{NOD}, relative to Mϕ_{C57}. Table 5 shows fatty acids production by Mϕ_{NOD}, relative to Mϕ_{C57}.

TABLE 3

| Lipid | Basal | | ^{b}IFNγ + LPS | |
|---|---|---|---|---|
| | (pmol/10⁶ Mϕ_{NOD}) | ^{a}Fold (Rel. to Mϕ_{C57}) | (pmol/10⁶ Mϕ_{NOD}) | ^{a}Fold (Rel. to Mϕ_{C57}) |
| 6-keto PGF₁α | 11.750 ± 3.436* | 6.306 ± 1.844 | 80.713 ± 5.252^{Δ} | 2.785 ± 0.181^{Δ} |
| TXB₂ | 6.619 ± 0.297^{†} | 1.529 ± 0.069 | 10.143 ± 0.328 | 0.936 ± 0.030 |
| PGD₂ | 0.278 ± 0.037 | 0.696 ± 0.092 | 0.630 ± 0.060^{†} | 0.025 ± 0.002 |
| 8-Iso PGF₂α | 1.951 ± 0.160^{Δ} | 23.958 ± 1.967 | 6.312 ± 0.284^{Δ} | 27.695 ± 1.247 |
| 5-IPFα-VI | 0.440 ± 0.078^{†} | 1.764 ± 0.312 | 0.682 ± 0.012^{#} | 2.111 ± 0.036 |
| PGE₂ | 1.607 ± 0.132 | 1.831 ± 0.150 | 165.684 ± 6.148^{#} | 2.235 ± 0.083 |
| PGA₂ | 0.853 ± 0.121^{#} | 2.257 ± 0.320 | 15.176 ± 0.618^{†} | 1.880 ± 0.077 |
| 15-deoxy-Δ12,14-PGJ₂ | 0.515 ± 0.048^{†} | 2.854 ± 0.268 | 0.992 ± 0.068^{#} | 2.341 ± 0.16 |
| LTD₄ | 0.257 ± 0.044^{¥} | 5.273 ± 0.908 | 0.154 ± 0.011^{∂} | 3.017 ± 0.216 |
| LTC₄ | 0.053 ± 0.026^{†} | 2.940 ± 1.423 | 0.043 ± 0.013 | 1.731 ± 0.536 |
| LTE₄ | 0.178 ± 0.067^{†} | 2.52 ± 0.946 | 0.069 ± 0.003 | 0.492 ± 0.023 |
| LTB₄ | 0.586 ± 0.023 | 1.602 ± 0.062 | 0.690 ± 0.048 | 1.236 ± 0.087 |
| 20-HETE | 22.803 ± 1.552 | 0.717 ± 0.049 | 22.644 ± 0.847 | 0.781 ± 0.029 |
| 15-HETE | 4.051 ± 0.245 | 1.020 ± 0.062 | 10.709 ± 0.366 | 1.211 ± 0.041 |
| 12-HETE | 40.970 ± 0.759 | 1.398 ± 0.026 | 48.677 ± 0.362^{†} | 1.441 ± 0.011 |
| 5-HETE | 4.655 ± 0.068 | 0.964 ± 0.014 | 6.573 ± 0.074 | 1.139 ± 0.013 |
| (±)14,15-DHET | 1.875 ± 0.034 | 0.989 ± 0.018 | 2.085 ± 0.150 | 1.196 ± 0.086 |
| (±)11,12-DHET | 0.479 ± 0.039 | 1.320 ± 0.107 | 0.606 ± 0.047 | 1.450 ± 0.114 |
| (±)8,9-DHET | 0.843 ± 0.054 | 1.815 ± 0.117 | 1.191 ± 0.049^{†} | 2.124 ± 0.087 |

TABLE 4

| Lipid | Basal | | ^{b}IFNγ + LPS | |
|---|---|---|---|---|
| | (pmol/10⁶ Mϕ_{NOD}) | ^{a}Fold (Rel. to Mϕ_{C57}) | (pmol/10⁶ Mϕ_{NOD}) | ^{a}Fold (Rel. to Mϕ_{C57}) |
| PGF₂α | 0.846 ± 0.134 | 0.421 ± 0.067 | 2.518 ± 0.152 | 0.321 ± 0.019 |
| PGE₁ | 0.190 ± 0.082 | 1.572 ± 0.678 | 28.501 ± 0.901^{†} | 1.884 ± 0.060 |
| Resolvin D2 | 0.0185 ± 0.004 | 0.070 ± 0.016 | 0.0174 ± 0.005 | 0.031 ± 0.01 |
| Resolvin D1 | 0.160 ± 0.010 | 1.410 ± 0.089 | 0.176 ± 0.011 | 1.533 ± 0.098 |
| Lipoxin A4 | 0.102 ± 0.045 | 0.581 ± 0.256 | 0.143 ± 0.043 | 0.774 ± 0.232 |
| (±)14,15-EET | 0.539 ± 0.007 | 0.766 ± 0.01 | 0.863 ± 0.044 | 1.051 ± 0.053 |
| (±)8,9-EET | 0.423 ± 0.050 | 1.294 ± 0.153 | 0.978 ± 0.096 | 1.771 ± 0.175 |

TABLE 5

| Lipid | Basal | | ^{b}IFNγ + LPS | |
|---|---|---|---|---|
| | (pmol/10⁶ Mϕ_{NOD}) | ^{a}Fold (Rel. to Mϕ_{C57}) | (pmol/10⁶ Mϕ_{NOD}) | ^{a}Fold (Rel. to Mϕ_{C57}) |
| EPA | 134.545 ± 2.625 | 0.936 ± 0.018 | 209.022 ± 2.095 | 1.365 ± 0.014 |
| DHA | 769.849 ± 9.955 | 0.740 ± 0.010 | 902.551 ± 13.428 | 0.886 ± 0.013 |
| AA | 5355.159 ± 244.850 | 0.895 ± 0.041 | 7715.520 ± 61.79 | 0.950 ± 0.008 |

Sphingolipids. As our earlier studies revealed that stress-induced β cell death is associated with increases in various proapoptotic ceramides (CMs) (17, 23, 24), Applicants assessed sphingolipids production by Mϕ_{NOD}. Applicants determined that several CM species (C16:0, C22:0, C24:1, C24:0) are higher in Mϕ_{NOD} under both basal and classical activation, relative to Mϕ_{C57} (FIGS. 8A and 8B). Some monohexyl CM (MHCM) species are decreased in MNOD, relative to Mϕ_{C57} - in particular, C16:0-MHCM (FIGS. 8C and 8D). Several sphingomyelin (SM) species (C18:1, C18:0, C20:0, C22:0, and C24:1) were elevated under basal conditions, with the 16:0 species decreasing in the Mϕ_{NOD}, relative to Mϕ_{C57} (FIGS. 8E and 8F). The only significant difference under classical activation was an increase in the C24:1-SM in Mϕ_{NOD}, relative to Mϕ_{C57}. Among the CM-1-phosophate (C1P) species, C22:0 was lower and C24:0 higher under basal conditions and 16:0 higher under classical activation in Mϕ_{NOD}, relative to Mϕ_{C57} (FIGS. 8G and 8H). Although little is known as to the chain length specificity of C1P in driving inflammatory responses, the C16:0 species is usually associated with inflammatory responses, induction of inflammatory eicosanoid biosynthesis, and Mϕ migration.

Collectively, these findings suggest that the spontaneous diabetes-prone NOD is inherently in a heightened inflammatory state, as reflected by the higher abundances of proinflammatory lipids and higher iPLA₂β mRNA (C57, 1.00 ± 0.07; NOD, 1.83 ± 0.05, P < 0.001, n = 3/group). Reduction in iPLA₂β expression in NOD mitigates T1D parameters and favors M2-Mϕ phenotype

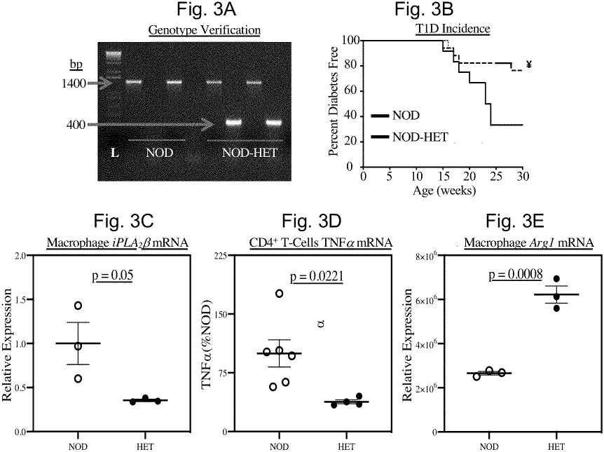

As the elevated lipids in Mϕ_{NOD} can be generated in an iPLA₂β-dependent manner, Applicants examined the consequences of reduced iPLA₂β expression on T1D development by comparing NOD and NOD-HET littermates. Genotype was verified by PCR analyses (FIG. 3A), which generated the expected product sizes of 1400 bp for NOD and 1400 bp and 400 bp for NOD-HET. Blood glucose monitoring revealed approximately 75% T1D incidence in NOD (FIG. 3B). In contrast, approximately 80% of the NOD-HET remained diabetes free, and this was accompanied by reduced iPLA₂β (^{~}65%) (FIG. 3C) and TNF-α production by CD4⁺ T cells (FIG. 3D) and higher M2 marker, Arg1 (FIG. 3E), relative to NOD. Furthermore, insulitis was reduced in the NOD-HET (20%-24%) relative to NOD (49%-56%). These findings support a link between iPLA₂β, Mϕ_{NOD} polarization, and T1D development, raising the importance of identifying the iDLs contributing to T1D development.

### Reduced iPLA₂β Expression Mitigates Mϕ_{NOD} Production of Select Proinflammatory Lipids

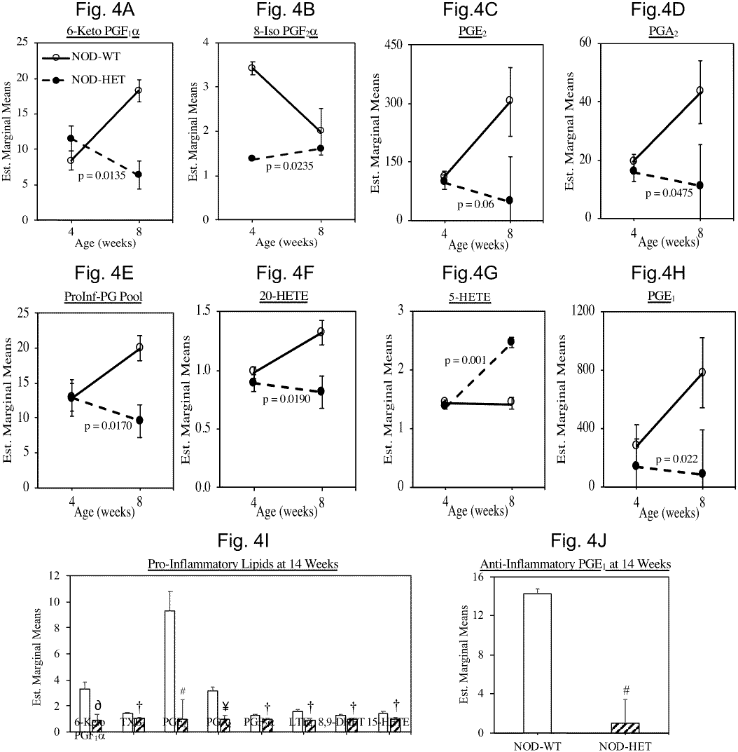

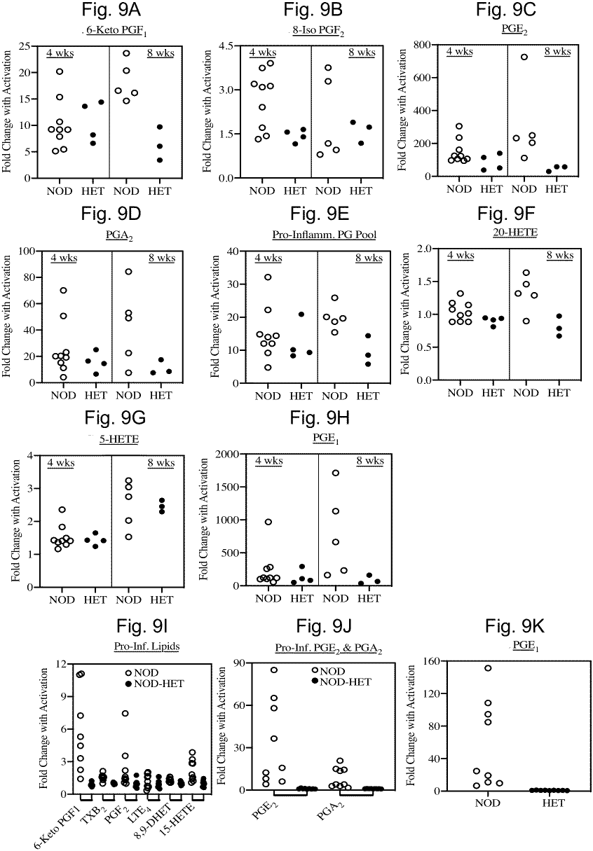

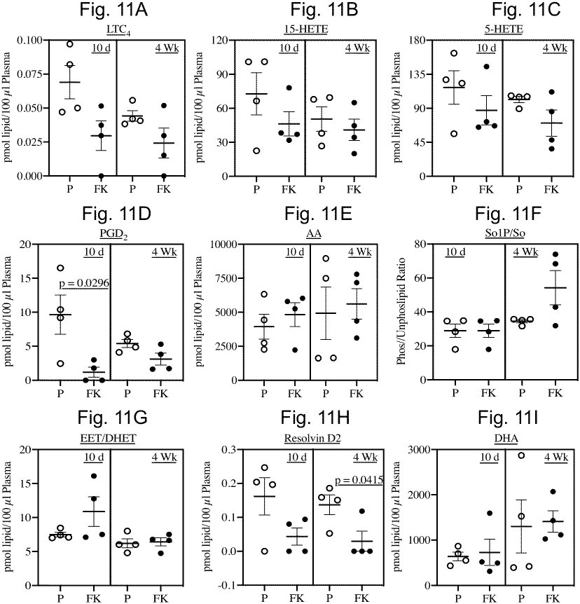

Eicosanoids and SPMs. In view of the above observations, Applicants examined whether decreased iPLA₂β expression would mitigate production of proinflammatory lipids by Mϕ between 4 and 8 weeks of age. Production of lipids by Mϕ_{NOD} and Mϕ_{NOD-HET} under classical activation was not significantly different at 4 weeks of age (FIG. 4), with the exception of 8-Iso PGF₂α, which was higher from Mϕ_{NOD}, relative to Mϕ_{NOD-HET}. Between 4 and 8 weeks of age, classical activation resulted in lower production of several proinflammatory lipids (6-keto PGF₁α, 8-Iso PGF₂α, PGE₂, PGA₂, total proinflammatory pool, and 20-HETE) by Mϕ_{NOD-HET} (FIGS. 4A-4F), relative to Mϕ_{NOD}. However, production of proinflammatory 5-HETE by Mϕ_{NOD-HET} was higher (FIG. 4G) and antiinflammatory was PGE₁ lower (FIG. 4H), relative to production by Mϕ_{NOD}. Interestingly by 14 weeks of age, the production of eicosanoids by Mϕ_{NOD} and Mϕ_{NOD-HET} was dramatically reduced, but production of several of the same proinflammatory PGs, LTE₄, (±) 8,9-DHET, and 15-HETE by Mϕ_{NOD} remained significantly higher, relative to Mϕ_{NOD-HET} (FIG. 4I). Moreover, production of PGE₁ by Mϕ_{NOD} continued to be higher, relative to Mϕ_{NOD-HET} (FIG. 4J; absolute fold increases from independent measures of select lipids are presented in FIG. 9). All other eicosanoids, SPMs, and fatty acids were not significantly affected between 4 and 14 weeks (Tables 5 and 6). These findings reveal that production of select proinflammatory eicosanoids is modulated by iPLA₂β in an age-dependent manner, before the development of hyperglycemia (FIG. 10). Table 5 shows classically-activated eicosanoids production by Mϕ_{NOD} and Mϕ_{NOD-HET}. Table 6 shows classically-activated fatty acids production by Mϕ_{NOD} and Mϕ_{NOD-HET}.

TABLE 5

| Lipid | Mϕ_{NOD} | | | Mϕ_{NOD-HET} | | |
|---|---|---|---|---|---|---|
| | 4 weeks (n = 9) | 8 weeks (n = 5) | 14 weeks (n = 9) | 4 weeks (n = 4) | 8 weeks (n = 3) | 14 weeks (n = 9) |
| TXB₂ | 1.568 ± 0.094 | 1.730 ± 0.134 | 1.415± 0.091 | 1.438 ± 0.121 | 1.510 ± 0.173 | 1.026 ± 0.091 |
| 5-iPF₂α-VI | 1.566 ± 0.168 | 1.429 ± 0.289 | 1.102 ± 0.057 | 1.059 ± 0.217 | 1.165 ± 0.373 | 1.034 ± 0.057 |
| 15-deoxy-Δ12,14-PGJ₂ | 1.937 ± 0.199 | 2.546 ± 1.195 | UD | 1.089 ± 0.257 | 1.130 ± 1.542 | UD |
| PGF₂α | 3.270 ± 0.369 | 2.863 ± 0.753 | 1.300 ± 0.090 | 1.834 ± 0.477 | 2.877 ± 0.972 | 0.965 ± 0.09 |
| Resolvin D1 | 1.056 ± 0.100 | 1.426 ± 0.196 | 0.940 ± | 1.003 ± 0.129 | 1.271 ± 0.253 | 0.981 ± 0.019 |
| LTC₄ | 1.414 ± 0.576 | 1.241 ± 0.212 | UD | 1.376 ± 0.744 | 1.091 ± 0.274 | UD |
| LTE₄ | 0.547 ± 0.169 | 1.651 ± 0.449 | 1.579 ± 0.188 | 0.559 ± 0.218 | 1.133 ± 0.580 | 0.899 ± 0.188 |
| LTB₄ | 1.185 ± 0.070 | 1.092 ± 0.232 | UD | 1.266 ± 0.090 | 1.311 ± 0.300 | UD |
| (±)14,15-DHET | 1.096 ± 0.063 | 0.949 ± 0.094 | 1.003 ± 0\|04 | 0.949 ± 0.094 | 1.058 ± 0.122 | 0.958 ± 0.04 |
| (±)11,12-DHET | 1.203 ± 0.100 | 2.128 ±0.566 | 1.105 ± 0.04 | 1.097± 0.129 | 1.135 ± 0.731 | 1.023 ± 0.04 |
| (±)8,9-DHET | 1.415 ± 0.061 | 1.696 ± 0.576 | 1.254 ± 0.089 | 0.987 ± 0.079 | 1.202 ± 0.744 | 0.973 ±0.089 |
| 15 HETE | 2.768 ± 0.088 | 4.646 ± 0.765 | 1.408 ± 0.142 | 2.177 ± 0.114 | 3.052 ± 0.987 | 0.952 ± 0.142 |
| 12 HETE | 1.205 ± 0.028 | 1.888 ± 0.378 | 1.113 ± 0.111 | 1.259 ± 0.037 | 1.662 ± 0.487 | 0.907 ± 0.111 |

TABLE 6

| Lipid | MΦ_{NOD} | | | | | |
|---|---|---|---|---|---|---|
| | 4 weeks (n = 9) | | 8 weeks (n = 5) | | 14 weeks (n = 9) | |
| EPA | 1.568 | ± 0.061 | 1.568 | ± 0.061 | 1.174 | ± 0.261 |
| DHA | 1.183 | ± 0.016 | 1.120 | ± 0.125 | 0.953 | ± 0.064 |
| AA | 1.436 | ± 0.053 | 2.219 | ± 0.238 | 0.766 | ± 0.265 |
| | MΦ_{NOD-HET}- | | | | | |
| | 4 weeks (n = 4) | | 8 weeks (n = 3) | | 14 weeks (n = 9) | |
| EPA | 1.180 | ± 0.071 | 1.744 | ± 0.108 | 1.042 | ± 0.261 |
| DHA | 1.052 | ± 0.020 | 1.136 | ± 0.161 | 0.946 | ± 0.064 |
| AA | 1.313 | ± 0.068 | 2.214 | ± 0.307 | 0.856 | ± 0.265 |

Sphingolipids. Though classical activation induced changes in the various sphingolipid classes, there were no significant differences in the total pools of CMs, monohexosyl CMs, SMs, CM-1Ps, or sphingosines between MΦ_{NOD} and MΦ_{NOD-HET} (Table 7). These findings suggest that iPLA₂β-mediated sphingolipid production by MΦ during the prediabetic phase may not be important contributors to T1D development.

TABLE 7

| Lipid | MΦ_{NOD} | | | | | |
|---|---|---|---|---|---|---|
| | 4 weeks (n = 9) | | 8 weeks (n = 5) | | 14 weeks (n = 9) | |
| Total CMs | 3.083 | ± 1.364 | 1.937 | ± 0.188 | 1.264 | ± 0.185 |
| Total MHCMs | 1.533 | ± 0.279 | 2.357 | ± 0.469 | 1.485 | ± 0.290 |
| Total SMs | 0.961 | ± 0.109 | 1.349 | ± 0.147 | 1.330 | ± 0.222 |
| Total C-1-Ps | 0.936 | ± 0.083 | 1.021 | ± 0.501 | 0.731 | ± 0.124 |
| So1P/So1 | 47.218 | ± 6.420 | 56.885 | ± 9.960 | | UD |
| | MΦ_{NOD-HET} | | | | | |
| | 4 weeks (n = 4) | | 8 weeks (n = 3) | | 15 weeks (n = 9) | |
| Total CMs | 1.156 | ± 1.760 | 1.496 | ± 0.242 | 1.515 | ± 0.278 |
| Total MHCMs | 0.999 | ± 0.322 | 2.661 | ± 0.542 | 1.590 | ± 0.435 |
| Total SMs | 1.014 | ± 0.126 | 1.337 | ± 0.169 | 1.533 | ± 0.333 |
| Total C-1-Ps | 0.635 | ± 0.096 | 1.922 | ± 0.578 | 0.749 | ± 0.186 |
| So1P/So1 | 47.753 | ± 8.288 | 51.256 | ± 12.859 | | UD |

### Select Plasma Lipid Changes Are Associated With iPLA₂β Inhibition or Expression

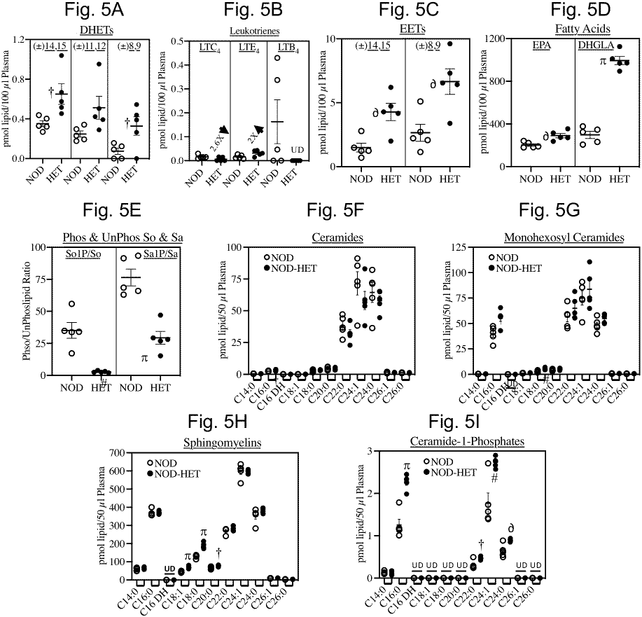

NOD versus NOD-HET. To determine if inhibition of iDL production can also be evidenced in circulating levels of lipids, Applicants assessed plasma lipidome of NOD and NOD-HET through 14 weeks of age (prediabetic phase). At 4 and 8 weeks of age, no significant differences in eicosanoids, sphingolipids, or fatty acids were noted between the NOD and NOD-HET (data not shown). At 14 weeks of age, proinflammatory DHET abundance was low but higher in the NOD-HET, relative to NOD (FIG. 5A). Among the proinflammatory LTs, LTC₄ was reduced 2.6-fold, its precursor LTE₄ increased 2-fold, and LTB₄ was absent in NOD-HET, relative to NOD (FIG. 5B). In contrast, the abundance of antiinflammatory epoxyeicosatrienoic acids (EETs) were greater and significantly higher in the NOD-HET, relative to NOD (FIG. 5C). Moreover, the abundance of EPA and di-homo-γ-linolenic acid (DHGLA) was higher in NOD-HET by 14 weeks of age, as compared with NOD (FIG. 5D). Furthermore, the ratios of phosphorylated to nonphosphorylated sphingosine (So1P/So) and sphinganine (Sa1P/Sa) were lower in the NOD-HET by 14 weeks of age, relative to NOD (FIG. 5E). Analyses of plasma CM sphingolipids revealed a select increase in CM C16:0 in NOD-HET, relative to NOD at 14 weeks of age (FIG. 5F). However, multiple monohexosyl CMs (FIG. 5G), SMs (FIG. 5H), and CM-1Ps (FIG. 5I), including the C16:0 species, were increased in the NOD-HET, relative to NOD.

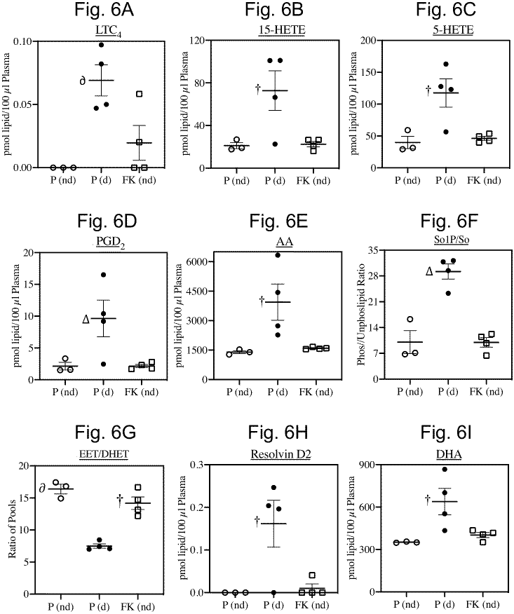

Next, to determine if a proinflammatory landscape persists until T1D onset, Applicants performed lipidomic analyses with plasma from FKGK18- and PBS-T-treated NOD (starting at 10 days). The analyses comparing PBS-T-treated NOD that did not become diabetic (P [nd]), vehicle-treated NOD that became diabetic (P [d]), and FKGK18-treated (from 10 days of age) NOD that did not turn diabetic (FK [nd]) revealed significantly greater abundance of proinflammatory LTC₄, 15-HETE, 5-HETE, PGD₂, and AA in the plasma from diabetic PBS-T-NOD, in comparison with nondiabetic PBS-T- or FKGK18-treated NOD (FIGS. 6A-6E, respectively). Furthermore, the ratio of So1P/So was higher in diabetic PBS-T-NOD, in comparison with nondiabetic PBS-T- or FKGK18-treated NOD (FIG. 6F). In contrast, the ratio of antiinflammatory EET to proinflammatory DHET pools was reduced in diabetic PBS-T-NOD, in comparison with nondiabetic PBS-T- or FKGK18-treated NOD (FIG. 6G). Surprisingly, SPM resolvin D2 (FIG. 6H) and its fatty acid source DHA (FIG. 6I) were significantly higher in the diabetic group, in comparison with either nondiabetic group. Comparison of PBS-T- and FKGK18-treated mice that developed T1D revealed no differences between the two groups (initiated at 10 days or 4 weeks of age), with the exception of a decrease in PGD₂ in the FKGK18 group, relative to the PBS-T (d) groups (FIG. 11). Collectively, these analyses reveal select changes in iPLA₂β-dependent plasma lipid profiles that may be important indicators of T1D development.

### Adoptive Transfer of Peritoneal iPLA₂β-deficient MΦ Reduces T1D Incidence

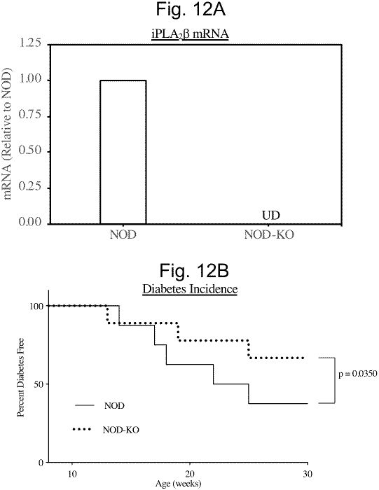

Adoptive transfer of M2-MΦ_{NOD} has been reported to reduce T1D incidence in the NOD. Using an analogous approach, Applicants performed an adoptive transfer study using peritoneal MΦ isolated from NOD and NOD.iPLA2β^{-/-} (KO) mice, rationalizing that the KO MΦ are analogous to M2-MΦ. Applicants found that T1D incidence in NOD administered the KO MΦ was significantly reduced, relative to the mice administered NOD MΦ (FIG. 12). These studies support the ability of peritoneal MΦ to infiltrate the islets and participate in the pathogenic process of T1D and support the idea that this can be mitigated when MΦ-iPLA₂β is reduced.

### Plasma Lipidome of Subjects at High Risk for Developing T1D

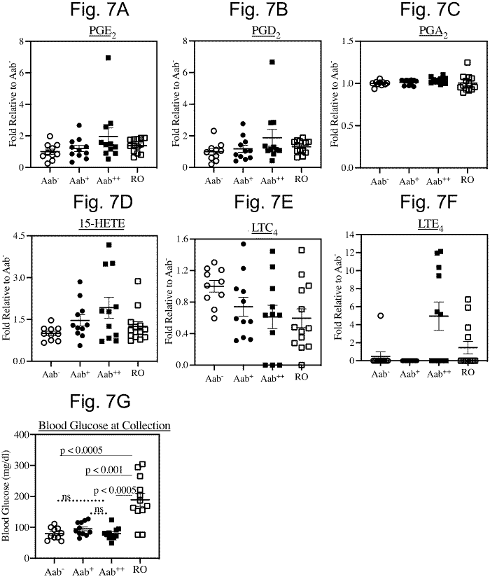

To determine if a similar lipid signature is evident in human subjects at high risk for developing T1D, plasma samples from nondiabetic (normoglycemic) autoantibody negative (Aab⁻), one Aab-positive (Aab⁺), or 2 Aab-positive (Aab⁺⁺) and recent-onset (RO, TID duration < 3.4 months) subjects were processed for lipidomics analyses (FIG. 7). The subjects were a mixture of male and female children, between 9 and 15 years old, where no significant differences in prevalence between the sexes are reported. These assessments identified increases in PGE₂, PGD₂, PGA₂, 15-HETE, and LTE₄, and a decrease in precursor LTC₄, in the Aab⁺⁺ group that were significant (P < 0.05) or approached significance (FIGS. 7A-7F), as reflected by Pearson, Kendall, and Spearman rank order correlation analyses (Table 1). Notably, differences recorded in the Aab⁺⁺ subjects occurred in the absence of hyperglycemia (FIG. 7G). Of import, these select proinflammatory eicosanoids exhibited a similar stepwise profile: Aab⁻ < Aab⁺ < Aab⁺⁺, with the RO group trending back to Aab⁻ levels. Taken together with the observations in the NOD models, these findings are consistent with a heightened select proinflammatory iDLs landscape in human subjects at high risk for developing T1D.

TABLE 8

| | Pearson’s R | | Kendall’s Tau | | Spearman’s Rho | |
|---|---|---|---|---|---|---|
| Lipid | Correlation | Sig. | Correlation | Sig. | Correlation | Sig. |
| PGE₂ | 0.337 | 0.059 | 0.260 | 0.064 | 0.337 | 0.059 |
| PGD₂ | 0.306 | 0.089 | 0.241 | 0.087 | 0.304 | 0.090 |
| PGA₂ | 0.440 | 0.012 | y0.338 | 0.016 | 0.425 | 0.015 |
| 15-HETE | 0.423 | 0.016 | 0.294 | 0.037 | 0.351 | 0.049 |
| LTC₄ | -0.397 | 0.025 | -0.300 | 0.033 | -0.372 | 0.036 |
| LTE₄ | 0.510 | 0.062 | 0.481 | 0.055 | 0.533 | 0.050 |

### Construction of NOD.PLA2G6^{-null}/Srvem Mice

NOD breeding pairs were obtained from The Jackson Laboratory, and only female progeny, with expected 80% diabetes incidence, were used in experiments. NOD.iPLA₂ß^{+/-} (NOD-HET) were generated by breeding male NOD with iPLA₂β-deficient (KO) female C57BL/6J. The investigator-induced-null PLA2G6 allele was congenically introgressed into the NOD genome by first generating F1 hybrids from outcrosses of C57BL/6J with NOD. These F1 hybrid females were backcrossed to NOD males, and the female progeny of each successive generation were backcrossed to NOD males for a total of 10 generations. To eliminate contaminating chromosomal segments, genotyping was performed by PCR amplification of 94 polymorphic microsatellite primers (Invitrogen) covering all 19 autosomes for the first 6 generations. By N6, mice were homozygous for NOD genome at all loci, except those in tight linkage with PLA2G6 on chromosome 15. From N6 until N10, genotyping was performed with markers on chromosome 15 to ensure transmission of the nonfunctional PLA2G6 allele, allowing for mice with the smallest possible congenic segment to be bred. At generation N10, these marker-assisted or speed congenic mice were intercrossed to generate mice that were homozygous for the PLA2G6^{-null} allele. These mice were then bred to generate NOD-WT (NOD) and NOD.iPLA2ß^{+/-} (NOD-HET) littermates used in subsequent studies.

### NOD Genotyping

Prior to experimentation, the mice were genotyped using the following primers (5′-3′) with expected product sizes: (sense/antisense: AGCTTCAGGATCTC-ATGCCCATC (SEQ ID NO: 1)/CTCCGCTTCTCGTCCCTCATGGA (SEQ ID NO: 2), 1400 bp; MaExAS/Neo; GGGGCCTCAGACTGGGA-ATC (SEQ ID NO: 3)/TCGCCTTCTATCGCCTTCTTGAC (SEQ ID NO: 4), 400 bp). Data from each genotype were compared against their corresponding WT littermates. The mice were maintained with a standard light/dark cycle with ad libitum access to food and water.

### Animal Treatment, Monitoring, and Assessments

Blood glucose levels were measured weekly via tail vein blood draw (2 µL) with Breeze 2 Blood Glucose Monitoring System (Bayer HealthCare). Diabetes incidence was based on 2 consecutive blood glucose readings ≥ 275 mg/dL, at which time the mouse was euthanized. Experimental groups included the following: (a) FKGK18, a reversible selective inhibitor of iPLA₂β (36) was administered via i.p. injection 3 times/week to NOD at 20 mg/kg body weight starting at 10 days, 4 weeks, or 8 weeks of age for up to 30 weeks, and mice receiving i.p. PBS + 5% Tween 80 (PBS-T) with the same dosing schedule were included as a vehicle control group; (b) NOD were treated with PBS-T or FKGK18 from 10 days to 14 weeks of age and then monitored for 30 weeks; (c) NOD and NOD-HET littermates were monitored for up to 30 weeks of age; and (d) NOD and NOD-HET mice were sacrificed at 4, 8, or 14 weeks of age for lipidomics analyses. For insulin measurements, blood was collected at sacrifice (nonfasting) into BD Microtainer Tubes with serum separator and processed for ELISA (Mercodia Kit). Other assessments included i.p. glucose tolerance test (IPGTT), islet infiltration, immunofluorescence analyses, β cell area, urine PGEM analyses, and CD4+ T cell assays. Pancreas section and islet images were captured on an Olympus IX81 microscope using cellSens Dimension software and analyzed using ImageJ software (NIH).

### Isolation and Activation of Peritoneal MΦ

Mice were euthanized by CO₂ inhalation and cervical dislocation. Peritoneal MΦ were obtained by filling the peritoneal cavity with cold 5 mL PBS containing 2% FBS, massaging gently, and withdrawing the cell-containing solution. Classical activation (IFN-y + LPS) experiments were performed with freshly isolated and expanded peritoneal MΦ. Briefly, MΦ were treated with 15 ng/mL recombinant IFN-γ (R&D Systems, 485-MI-100) for 8 hours in growth medium followed by addition of 10 ng/mL ultrapure LPS (InvivoGen, tlrl-3pelps) and incubated for 16 hours at 37° C. or IL-4 (R&D Systems, 404-ML-010) for 16 hours in growth medium. Naive MΦ, which received no activation stimuli, were maintained in growth medium with no additional treatment. Subsequently, the media and MΦ were collected for analyses of eicosanoid and sphingolipid classes of lipids, respectively.

### MΦ mRNA Target Analyses

MΦ cultured in 60 mm nontissue culture-treated dishes were lysed in 1 mL of TRIzol (Invitrogen, 15596-026). Total RNA was prepared and purified using RNeasy Mini Kits (QIAGEN, 74104), and 1 µg RNA was converted to cDNA using the Superscript III first-strand synthesis system (Invitrogen, 18080-051), according to manufacturer’s instructions. The cDNA was diluted 10-fold and used as template in conventional or quantitative PCR (qPCR). cDNA transcripts were amplified with the following forward/reverse primers (5′-3′) at Tₘ: PLA2G6_qRT, GGCAGAAGTGGACACCCCAA (SEQ ID NO: 5)/CATGGAGCTCAGGATGAACGC (SEQ ID NO: 6), 60° C.; msARG1_qRT, AGCACTGAG-GAAAGCTGGTC (SEQ ID NO: 7)/CAGACCGTGGGTTCTTCACA (SEQ ID NO: 8), 60° C.; and 18S-qRT, CGCTTCCTTACCTGGTTGAT (SEQ ID NO: 9)/ TCCCTCTCCGGAATCGAA (SEQ ID NO: 10), 60° C. qPCR was carried out using SYBR Select Mastermix (Invitrogen, 4472908) according to manufacturer’s instructions using 18S as an internal control. Relative gene expression levels were determined using the 2^{-ΔΔCt} method.

### Lipidomics Analyses

Eicosanoids preparation. Eicosanoids were extracted using a modified extraction process. Plasma (150 µL) was combined with 850 µL of liquid chromatography-mass spectrometry H₂O, followed by the addition of an internal standard (IS) mixture. For media analysis, IS was added to media from cells (2 mL). Eicosanoid IS was comprised of 10% methanol (100 µL and 200 µL, respectively, for plasma and media), glacial acetic acid (5 µL and 10 µL, respectively, for plasma and media), and internal standard (20 µL) containing the following deuterated eicosanoids (1.5 pmol/µL, 30 pmol total; all standards purchased from Cayman Chemicals): (d₄) 6-keto PGF₁α, (d₄) PGF₂α, (d₄) PGE₂, (d₄) PGD₂, (d₈) 5-HETE, (d₈) 12-HETE, (d₈) 15-HETE, (d₆) 20-HETE, (d₁₁) 8,9 epoxyeicosatrienoic acid, (d₈) 14,15 epoxyeicosatrienoic acid, (d₈) arachidonic acid, (d₅) eicosapentaenoic acid, (d₅) docosahexaenoic acid, (d₄) PGA₂, (d₄) LTB₄, (d₄) LTC₄, (d₄) LTD₄, (d₄) LTE₄, (d₅) 5(S),6(R)-lipoxin A4, (d₁₁) 5-iPF₂α -VI, (d₄) 8-iso PGF₂α, (d₁₁) (±) 14,15-DHET, (d₁₁) (±) 8,9-DHET, (d₁₁) (±) 11,12-DHET, (d₄) PGE₁, (d₄) thromboxane B2, (d₆) dihomo-γ-linoleic acid, (d₅) resolvin D2, (d₅) resolvin D1, (d₅) maresin 2, and (d₅) resolvin D3. Samples and vial rinses (5% MeOH; 2 mL) were applied to Strata-X SPE columns (Phenomenex), previously washed with methanol (2 mL) and then dH2O (2 mL). Eicosanoids eluted with isopropanol (2 mL) were dried in vacuuo and reconstituted in EtOH/dH₂O (50:50; 100 µL) before analysis.

Sphingolipids preparation. Cell pellets and plasma (50 µL) were extracted using a modified Bligh Dyer extraction. Samples were spiked with 250 pmol of C1P, SM, CM, and monohexosyl CM (d18:1/12:0 species), and sphingansine, sphinganine, sphingasine-1-phosphate, sphinganine-1-phosphate (d17:0 sphinganine/d17:1 sphingosine) as internal standard (Avanti Polar Lipids). Among the sphingolipids analyzed were CMs (C14:0, C16:0, C18:1, C18:0, C20:0, C22:0, C24:1, C24:0, C26:1, C26:0), monohexyl CMs (C14:0, C16:0, C18:1, C18:0, C20:0, C22:0, C24:1, C24:0, C26:1, C26:0), SMs (C14:0, C16:0, C18:1, C18:0, C20:0, C22:0, C24:1, C24:0, C26:1, C26:0), CM-1-phosphates (C14:0, C16:0, C18:1, C18:0, C20:0, C22:0, C24:1, C24:0, C26:1, C26:0), 18:1-sphingosine (C18:1-So) C18:1-sphingosine-1-phosphate (C18:1-So1P), and C18:1-sphinganine (C18:1-Sa) and C18:1-sphinganine-1-phosphate (C18:1-Sa1P).

Analysis of sphingolipids, eicosanoids, and fatty acids by ultra performance liquid chromatography-electrospray ionization-tandem mass spectrometry. Lipids in the samples were separated using 2 Shimadzu Nexera X2 LC-30AD pumps coupled to a SIL-30AC auto injector, coupled to a DGU-20A5R degassing unit. Sphingolipids, eicosanoids, and fatty acids were analyzed via mass spectrometry using an AB Sciex Triple Quad 5500 Mass Spectrometer. MRM transitions with corresponding declustering potentials, collision energies, entrance potentials, and collision cell exit potentials are shown in Tables 1 and 2. MΦadoptive transfer. Peritoneal MΦ were obtained from 8-week-old female NOD and NOD.iPLA₂β^{-/-} (NOD-KO) mice. The MΦ (2.75 × 10⁶) were administered i.p. to 8-week-old female NOD, and diabetes incidence was recorded through weekly blood glucose monitoring.

Human lipidome. Study subjects were recruited through Children’s Hospital of Wisconsin, and diagnosis of T1D was defined per World Health Organization criteria. All RO T1D subjects were positive for > 1 Aab and were drawn from subjects with histories of good glycemic control (HbA1c, 7.53% ± 0.28%). Subjects within the 3 nondiabetic (normoglycemic) sibling groups (Aab⁻, Aab⁺, Aab⁺⁺) were free of known infection at blood collection.

Significant difference in T1D incidence was determined by the Mantel-Cox test. For all other analyses, P values were determined using either 2-tailed Student’s t test (for analysis of 2 groups), multivariate analysis of variance (for analyses testing more than 1 outcome), time-course ANOVA (for temporal lipid analysis), or 1- or 2-way ANOVA (for tests including more than 1 sample group). To assess the relationship between selected eicosanoids in human plasma, the data were subjected to a linear regression analysis (Pearson, Kendall, and Spearman rank-order correlation) using only the Aab⁻, Aab⁺, and Aab⁺⁺ values, under the rationale that RO subjects are already diabetic and usually controlled with therapeutics. Statistical programs used were either SPSS or R; P < 0.05 was taken to indicate significant differences.

All animal experiments were conducted according to approved IACUC guidelines at UAB. Human study participants were recruited through the Children’s Hospital of Wisconsin, and samples were collected. IRB approval (CHW IRB 01-15) was granted for all analyses, and informed consent/assent was obtained from subjects or their parents/legal guardians. Acquisition and analyses of samples was approved by UAB (IRB-100813004).

1. An engineered macrophage comprising a genetic disruption in the gene encoding iPLA₂β in the engineered macrophage.

2. The engineered macrophage of claim 1, wherein the iPLA₂β gene disruption has been induced by CRISPR-Cas9.

3. The engineered immune cell of claim 1, wherein:
the disruption comprises disrupting the iPLA₂β gene at the DNA level,
the disruption is not reversible; or
the disruption is not transient.

4. The engineered macrophage of claim 1, wherein the macrophage is a peritoneal macrophage.

5. A pharmaceutical composition comprising the engineered macrophage of claim 1 and a pharmaceutically-acceptable carrier.

6. A method of treating Type 1 Diabetes in a subject, the method comprising administering a therapeutically effective amount of a pharmaceutical composition comprising an engineered macrophage comprising a genetic disruption in the gene encoding iPLA₂β in the engineered macrophage.

7. The method of claim 6, wherein the iPLA₂β gene disruption has been induced by CRISPR-Cas9.

8. The method of claim 6, wherein:
the disruption comprises disrupting the iPLA₂β gene at the DNA level,
the disruption is not reversible; or
the disruption is not transient.

9. The method of claim 6, wherein the engineered macrophage is a peritoneal macrophage.

10. The method of claim 6, wherein the pharmaceutical composition comprises a pharmaceutically-acceptable carrier.

11. The method of claim 6, wherein the pharmaceutical composition is administered intra-arterially, intravenously, intrapleurally, intravesicularly, by peritoneal injection, or orally.

12. The method of claim 6, wherein the pharmaceutical composition is administered at two or more time points, wherein the time points are separated by at least 24 hours.

13. The method of claim 6, wherein the subject is at risk of developing Type 1 Diabetes or exhibits symptoms of Type 1 Diabetes.
